(19) United States
(12) Patent Application Publication
(10) Pub. No.: US 2024/0173077 A1
(43) Pub. Date: May 30, 2024

(54) SMART SURGICAL INSTRUMENT SELECTION AND SUGGESTION

(71) Applicant: Warsaw Orthopedic, Inc., Warsaw, IN (US)

(72) Inventors: Jyotika Ghosh, Hyderabad (IN); Praveena Narayanabhatla, Hyderabad (IN); Bharathi Devi Chamana, Hyderabad (IN); Premkumar Rathinasabapathy Jagamoorthy, Hyderabad (IN); Vijaya Lakshmi Gunda, Hyderabad (IN)

(21) Appl. No.: 18/071,500

(22) Filed: Nov. 29, 2022

(51) Int. Cl. A61B 34/10; G16H 50/20; G16H 50/70

(52) U.S. Cl. CPC A61B 34/10; G16H 50/20; G16H 50/70; A61B 2034/105; A61B 2034/107

(57) ABSTRACT

A system and techniques are provided for suggesting a surgery plan and a surgical instrument selection. In some embodiments, the system may be configured to receive a set of inputs for a surgical procedure for a patient. Subsequently, the system may determine one or more potential plans for the surgical procedure based at least in part on the set of inputs and, in some implementations, a machine learning model. The system may then receive a selection of a plan from the one or more potential plans and determine a plurality of surgical instruments corresponding to the plan from the selection. Accordingly, the system may then be configured to provide an output that indicates the plurality of surgical instruments to load in a surgical tray. In some embodiments, the system may be configured to load the plurality of surgical instruments into the surgical tray.

### BACKGROUND

The present disclosure is generally directed to robotic-assisted surgeries, and relates more particularly to surgical instrument selection and suggestion for the robotic-assisted surgeries.

Surgical robots may assist a surgeon or other medical provider in carrying out a surgical procedure and/or may complete one or more surgical procedures autonomously. The surgical procedure(s) may be performed using one or more surgical instruments or tools. In some cases, a surgeon or other medical provider may manually select the one or more surgical instruments or tools prior to and for performing the surgical procedure(s). Additionally, the number of available surgical instruments or tools for performing the surgical procedure(s) is large, such that the surgeon or other medical provider may spend an inordinate amount of time manually selecting the one or more surgical instruments or tools, potentially prolonging the surgical procedure(s).

### BRIEF SUMMARY

Example aspects of the present disclosure include:

A system for suggesting a surgery plan and a surgical instrument selection, comprising: a processor; and a memory storing data for processing by the processor, the data, when processed, causes the processor to: receive a set of inputs for a surgical procedure for a patient; determine one or more potential plans for the surgical procedure based at least in part on the set of inputs and a machine learning model; receive a selection of a plan from the one or more potential plans; determine a plurality of surgical instruments corresponding to the plan from the selection; and provide an output that indicates the plurality of surgical instruments to load in a surgical tray.

Any of the aspects herein, wherein the data stored in the memory that, when processed causes the processor to provide the output that indicates the plurality of surgical instruments to load in the surgical tray further causes the system to: display, via a user interface, a suggestion of the plurality of surgical instruments to load in the surgical tray.

Any of the aspects herein, wherein the data stored in the memory that, when processed causes the processor to provide the output that indicates the plurality of surgical instruments to load in the surgical tray further causes the system to: load the plurality of surgical instruments into the surgical tray.

Any of the aspects herein, wherein the data stored in the memory that, when processed causes the processor to determine the one or more potential plans for the surgical procedure further causes the system to: compare the set of inputs for the surgical procedure with historical data of previously performed surgical procedures to determine the one or more potential plans based at least in part on a similarity index.

Any of the aspects herein, wherein the memory stores further data for processing by the processor that, when processed, causes the processor to: display, via a user interface, one or more similarity index values for each of the one or more potential plans.

Any of the aspects herein, wherein the historical data of previously performed surgical procedures comprises procedure and instrument flow and abnormalities, radiology images and annotations, demographic information, three-dimensional anatomical models, angles, positions, dimensions, implants used with respect to the three-dimensional models, instrument availability information, treatment plans, or a combination thereof, for the previously performed surgical procedures.

Any of the aspects herein, wherein the similarity index comprises a positional coordinate correlation, a deformation coefficient, demographic information, three-dimensional model, inventory stock matching of available surgical instruments, or a combination thereof.

Any of the aspects herein, wherein the memory stores further data for processing by the processor that, when processed, causes the processor to: provide a position suggestion for the surgical procedure for the patient corresponding to the plan from the selection.

Any of the aspects herein, wherein the memory stores further data for processing by the processor that, when processed, causes the processor to: receive one or more changes to the plan from the selection, wherein the plurality of surgical instruments to load in the surgical tray are determined based at least in part on the one or more changes.

Any of the aspects herein, wherein the memory stores further data for processing by the processor that, when processed, causes the processor to: provide a cut sectional view for the surgical procedure for the patient based at least in part on the set of inputs; and provide one or more cut sectional views for the one or more potential plans based at least in part on historical surgical data corresponding to the one or more potential plans, wherein the selection of the plan from the one or more potential plans is received based at least in part on a comparison of the cut sectional view for the surgical procedure and the one or more cut sectional views for the one or more potential plans.

Any of the aspects herein, wherein the memory stores further data for processing by the processor that, when processed, causes the processor to: receive feedback after the surgical procedure for the patient is performed based at least in part on the output that indicates the plurality of surgical instruments to load in the surgical tray.

Any of the aspects herein, wherein the feedback is used, in part, to train the machine learning model.

Any of the aspects herein, wherein the one or more potential plans are determined based at least in part on a disease state corresponding to the surgical procedure for a patient.

Any of the aspects herein, wherein the set of inputs for the surgical procedure comprises patient demographic data, one or more radiological images, pathology data, or a combination thereof.

Any of the aspects herein, wherein the output that indicates the plurality of surgical instruments to load in the surgical tray comprises an order for loading the plurality of surgical instruments in the surgical tray.

A system for suggesting a surgery plan and a surgical instrument selection, comprising: a processor; and a memory storing data for processing by the processor, the data, when processed, causes the processor to: receive a set of inputs for a surgical procedure for a patient; determine one or more potential plans for the surgical procedure based at least in part on the set of inputs; receive a selection of a plan from the one or more potential plans; determine a plurality of surgical instruments corresponding to the plan from the selection; and provide an output that indicates the plurality of surgical instruments to load in a surgical tray.

Any of the aspects herein, wherein the data stored in the memory that, when processed causes the processor to determine the one or more potential plans for the surgical procedure further causes the system to: compare the set of inputs for the surgical procedure with historical data of previously performed surgical procedures to determine the one or more potential plans based at least in part on a similarity index.

Any of the aspects herein, wherein the memory stores further data for processing by the processor that, when processed, causes the processor to: display, via a user interface, one or more similarity index values for each of the one or more potential plans.

A system for suggesting a surgery plan and a surgical instrument selection, comprising: a processor; and a memory storing data for processing by the processor, the data, when processed, causes the processor to: receive a set of inputs for a surgical procedure for a patient; determine one or more potential plans for the surgical procedure based at least in part on the set of inputs; receive a selection of a plan from the one or more potential plans; determine a plurality of surgical instruments corresponding to the plan from the selection; and load the plurality of surgical instruments into a surgical tray.

Any of the aspects herein, wherein the plurality of surgical instruments is loaded in the surgical tray according to an order for performing the surgical procedure for the patient based at least in part on the plan from the selection.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a diagram of a workflow according to at least one embodiment of the present disclosure;

FIG. 3 is a diagram of a system according to at least one embodiment of the present disclosure;

FIG. 4 is a flowchart of a method according to at least one embodiment of the present disclosure;

FIG. 5 is a flowchart of a method according to at least one embodiment of the present disclosure; and

FIG. 6 is a flowchart of a method according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In some surgical procedures (e.g., robotic-assisted surgeries), a minimally invasive procedure may be performed for the treatment of pathological fractures of the vertebral body (e.g., spine and associated elements) due to osteoporosis, cancer, benign lesions, or other ailments. For example, the minimally invasive procedure may include a corpectomy (e.g., a surgical procedure that involves removing all or part of the vertebral body, usually as a way to decompress the spinal cord and nerves), kyphoplasty (e.g., a surgical procedure used to treat a spinal compression fracture based on inserting an inflatable balloon tamp into a fractured vertebra to restore height to the collapsed vertebra), vertebroplasty (e.g., a procedure for stabilizing compression fractures in the spine based on injecting bone cement into vertebrae that have cracked or broken), radiofrequency ablation (e.g., a medical procedure in which part of the electrical conduction system of the heart, tumor, or other dysfunctional tissue is ablated using the heat generated from a medium frequency alternating current to treat a range of conditions, including chronic back and neck pain), or another procedure not explicitly listed herein. Additionally or alternatively, the surgical procedures described herein may more generally include spine surgeries, cranial surgeries, or another type of surgical procedure.

The surgical procedures may comprise a number of steps. In a first step for a pre-operative setup, specific instruments and accessories (e.g., for bone access, fracture reduction, stabilization, etc.) are arranged, and a position of the patient is decided (e.g., to reduce load on the fractured bone or other area of interest). A second step for imaging setup may include verifying different scans (e.g., magnetic resonance imaging (MRI) scans, computed tomography (CT) scans, etc.) to determine a best inclination to access the area of interest (e.g., fractured bone) and placement for other components for the surgical procedure (e.g., inflatable balloon, bone cement injection, current generating source, etc.). A third step may be performed for bone access, where a best incision location is determined and marked (e.g., with a surgical pen) and a biopsy (e.g., bone biopsy) may be taken to exclude the possibility of malignancy.

A fourth step may be performed for fracture reduction, where preparation on the insertion and positioning of components for the surgical procedure are determined and performed (e.g., directly under or proximal to the fracture zone). A fifth step may then include a fracture fixation, such as selecting a filler and volume gauge, preparing elements (e.g., package or bone cement) for insertion, delivering the elements to the area of interest (e.g., cement is delivered to a location until a cavity is filled), removing components (e.g., a cannula) used for the surgical procedure from the patient, and closing the incision. A sixth and last step may include determining indications and outcomes of the procedure (e.g., if the procedure was successful, such as successfully reducing pain or mending a fracture). In some cases, navigation technology may be used that allows for real-time visualization of the anatomy corresponding to the area of interest relative to a pre-operative plan. Additionally, the navigation may provide visibility that closes the loop on the execution of the pre-operative plan.

It is to be understood that the surgical procedures described herein may not include all of the steps described above or may include additional steps not listed. More generally, the surgical procedures may include presurgical planning of ensuring all the correct surgical instruments and disposables are ready for the surgical procedure, working with radiology imaging to ensure a correct scan format is used, and troubleshooting any communication issues with integrated third-party systems. Additionally, the surgical procedures may include a before surgery time to provide system setup and functional verification for the surgical procedure, a during surgery time for troubleshooting and resolving equipment and instruments issues and for providing real-time guidance and training to operating room staff, and a post-surgery time for checking and stowing any equipment used during the procedure and for reviewing case questions with the operating room staff.

In any of the examples of the surgical procedures described herein, a surgeon or other medical provider must choose the appropriate and correct medical instruments prior to and for performing the surgical procedures. For example, the surgeon or other medical provider may determine the appropriate and correct medical instruments based on the disease state for a given patient, which may depend on various factors, such as angle, position, depth, level of deterioration, size of tumor, etc. Once the target area and disease state are identified and determined, the surgeon may manually select the surgical instruments to perform the corresponding surgical procedure.

However, a large number of surgical instruments may be available for performing the surgical procedure. For example, more than 200 surgical instruments may be available for spine surgeries, and more than 100 surgical instruments may be available for cranial surgeries. Subsequently, the surgeon or other medical provider may take an inordinate amount of time (e.g., up to 20% of the entire surgical procedure time, which may equate to approximately 40-60 minutes in some cases) to select the surgical instruments manually. Additionally, many of the surgical instruments may have more than one tip or other interchangeable component, where the surgeon or other medical provider has to select the tip or other interchangeable component that fits into a verification divot of the corresponding surgical instrument, and then the surgeon or other medical provider must verify the surgical instrument manually. The process of verifying the interchangeable components for a corresponding surgical instrument may be performed for each instrument.

Additionally, the surgical instruments may not be well labeled to be handpicked blindly while performing surgery, impacting an ability of the surgeon or other medical provider from being able to pick the correct surgical instrument quickly and efficiently. Accordingly, the planning of the surgical procedure in an optimized manner may become cumbersome for the surgeons to perform, leading to longer surgical procedure planning and execution.

As described herein, a machine learning model (e.g., artificial intelligence (AI)-based learning model or algorithm) is provided for suggesting and/or auto-loading a surgery tray with needed surgical instruments for a surgical procedure to reduce surgery time. For example, the machine learning model may be utilized to provide suggestions for a surgery plan, provide suggestions for instrument selection, and/or autoload surgical instruments in an order based on various historical parameters of previously performed surgical procedures. In some embodiments, the machine learning model and associated techniques described herein may provide an efficient way to auto-suggest appropriate surgical instruments for a specific procedure to save a surgeon time and reduce a time of the corresponding procedure overall.

That is, a recommendation of surgical tools, instruments, implants, etc., and a procedure recommendation may be provided based on historical data of previously performed surgical procedures. For example, the historical data of the previously performed surgical procedures may include procedure and instrument flow and abnormalities (e.g., which surgical instruments were used, in which order the surgical instruments were used, any abnormalities that were present, etc.), radiology images and annotations for the surgical procedures (e.g., MRI scans, CT scans, X-rays, etc.), demographic information for a corresponding patient, instruments availability information (e.g., hospital available inventory data indicating which surgical instruments are or were available for use), three-dimensional (3D) anatomical model driven image analyses (e.g., powered by AI-based learning models), or a combination thereof, for the previously performed surgical procedures. Subsequently, a surgery instrument planning, preview, and/or autoloading of a surgical instruments tray in an order of which instruments are to be used during the surgical procedure may be provided (e.g., considering the abnormalities to reduce surgical time for image guided surgeries).

Embodiments of the present disclosure provide solutions to one or more of the problems of (1) prolonged surgical procedure durations, (2) increased exposure to anesthesia and/or radiation for a patient, and (3) higher chances of misdiagnoses or improperly performed surgical procedures. For example, the techniques described herein may shorten the instrument selection process of surgical procedures, which results in shorter procedure durations, may reduce a patient's anesthesia dosage and timing, reduces radiation exposure (e.g., to confirm implant positioning), and promotes faster recovery. Additionally, the techniques may be driven by an intelligent learning model that is normalized and optimized to meet clinical demand, thereby reducing the chances of misdiagnoses, and considering the complexity of the surgical procedures, the patient may benefit from both time and cost perspectives.

Additionally, predictive diagnostic decision-making may benefit the surgeon by providing a clear planning pathway and may provide the surgeon an opportunity to explore various options at a planning level, which can reduce unforeseen surprises during the procedure. Auto-evaluation of historical parameters extracted from procedures may also provide an excellent solution to analyze the historical procedures and draw inferences with clear insights. The autosuggestion of instruments may remove a cognitive burden during operating theater (OT) setup and pre-procedure planning and may provide faster workflow transitions (e.g., to navigation task). In some embodiments, the machine learning model provided herein may utilize a 3D model driven correlation to consider all aspects of an anatomical region of interest for properly analyzing critical structures (e.g., including any deformity) before providing the instrument suggestion to optimally fit a given surgical scenario.

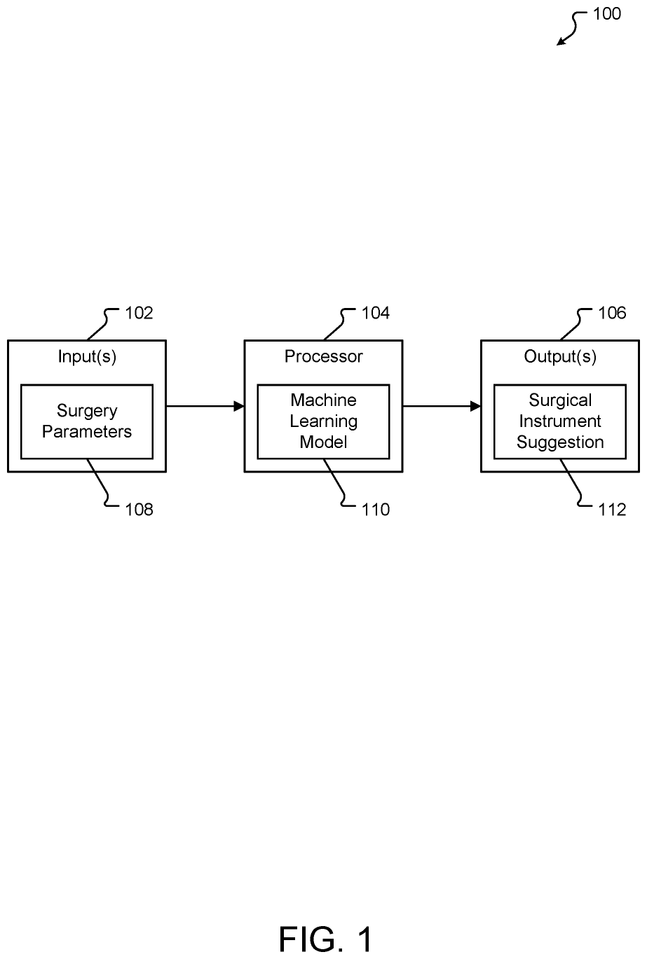

FIG. 1 is a block diagram of a system 100 according to at least one embodiment of the present disclosure. The system 100 may include one or more inputs 102 that are used by a processor 104 to generate one or more outputs 106. The processor 104 may be part of a computing device or different device. Additionally, the processor 104 may be any processor described herein or any similar processor. The processor 104 may be configured to execute instructions or data stored in a memory, which the instructions or data may cause the processor 104 to carry out one or more computing steps utilizing or based on the inputs 102 to generate the outputs 106.

As described herein, the inputs 102 may include a set of surgery parameters 108 for a surgical procedure for a patient. For example, the set of surgery parameters 108 may include patient demographic data, one or more radiological images, pathology data, or a combination thereof.

Subsequently, the processor 104 may use the set of surgery parameters 108 to predict an exact disease state for the patient and for the surgical procedure based on a machine learning model 110 (e.g., machine learning algorithm, AI-based algorithm or model, etc.). For example, the machine learning model 110 may be created based on available historical data of previously performed surgical procedures, which includes procedure and instrument flow and abnormalities (e.g., which surgical instruments were used, in which order the surgical instruments were used, any abnormalities that were present, etc.), radiology images and annotations (e.g., MRI scans, CT scans or images, X-rays, etc.), demographic information of the patients that underwent the previously performed surgical procedures, 3D anatomical models (e.g., indicating angles, positions, dimensions, etc. for the previously performed surgical procedures), instruments availability information (e.g., hospital available inventory data indicating which surgical instruments are or were available for use), or a combination thereof for each of the previously performed surgical procedures. In some embodiments, the machine learning model 110 may be continuously improved based on continuous feedback from surgeons after surgical procedures are completed.

In some embodiments, the processor 104 may use the machine learning model 110 to compare the set of surgery parameters 108 with the available historical data. Based on the comparison using the machine learning model 110, the processor 104 may generate a list of various closest matching surgical procedures (e.g., in relation to the surgical procedure for which the set of surgery parameters 108 are provided) to display to the surgeon (e.g., or other medical provider). The closest matching surgical procedures may be compared to the surgical procedure for which the set of surgery parameters 108 are provided and compared between each other with a similarity index that comprises a positional coordinate correlation (e.g., surgery anatomical position, implant location, etc.), a deformation coefficient, demographic similarity (e.g., body mass index (BMI) and/or other demographic information for the associated patients), 3D model similarity, inventory stock matching (e.g., whether same surgical instruments are available for the surgical procedure that were available and used for the closest matching surgical procedures), or a combination thereof. In some examples, different similarity index values for the different components of the similarity indexes of each of the closest matching surgical procedures may be displayed to the surgeon to indicate how similar each of the individual components are between the surgical procedure and the closest matching surgical procedures. Additionally or alternatively, an overall similarity index may be displayed indicating how similar the surgical procedure is in relation to each of the closest matching surgical procedures.

Subsequently, based on the similarity index(es), the surgeon can choose the closest possible previously executed surgery, which will help to map and to identify the surgical flow. As part of the identified surgical flow, the processor 104 may provide a surgical instrument suggestion 112 as part of the output(s) 106 (e.g., suggestion of which surgical instruments to use based on which surgical instruments were used for the chosen closest possible previously executed surgery). For example, the processor 104 may display (e.g., via a user interface) a suggestion of the surgical instruments to load in a surgical tray for the surgeon to perform the surgical procedure. Based on the chosen closest possible previously executed surgery, the processor 104 may also suggest what should be the position of the patient.

Based on these inputs (e.g., surgical flow, surgical instrument suggestion 112, position of the patient, etc.), the surgeon can edit or accept the plan corresponding to the closest possible previously executed surgery. In some embodiments, after the plan is confirmed and/or edited, the processor 104 may preload the surgical instruments (e.g., from within a surgical instrument depository storing a plurality of surgical instruments) and place the surgical instruments in a surgery tray in an order for the surgeon to use for the surgical procedure. Additionally or alternatively, the processor 104 may simply provide an output that indicates which surgical instruments to place or load in the surgical tray (e.g., the surgical instrument suggestion 112). The surgeon can then complete the surgery and provide the feedback back to the machine learning model 110 as part of a feedback loop, which will help to further mature the machine learning model 110.

Accordingly, as described herein, the machine learning model 110 may be developed based on the surgery parameters 108 (e.g., patient input data, such as radiology and physiology images and data) and previous surgical data of similar procedures (e.g., stored in a database), where the previous surgical data may include 3D models, angle and position, depth of implant and dimension, annotations, treatment plans, radiology diagnostic imaging, an implant used with respect to the 3D models, or a combination thereof for each of the similar procedures. The processor 104 may then use the machine learning model 110 to suggest one or more surgery plans to the surgeon, including the disease state for the patient (e.g., angle, depth, and position of the targeted area) based on the similar surgical data. In some examples, the suggested surgery plans may be suggested or displayed to the surgeon in a 3D model view.

Depending on the similar procedures and which surgical instruments are available at a hospital in which the surgical procedure is to be performed (e.g., a hospital inventory of surgical instruments), the processor 104 may present one or more similarity index(es) to the surgeon indicating a closest match of the available previous surgeries to the surgical procedure to be performed. For example, the similarity index(es) may be percentage(s) of how close different aspects of each similar procedure is to the surgical procedure to be performed, such as a similarity of percentage deterioration between the surgical procedures, a location similarity, an implant depth percentage, a disease correlation, or other comparable aspects between the similar procedures and the surgical procedure to be performed.

Based on the similarity index(es), the surgeon may select one of the similar procedures to follow. In some embodiments, after making the selection, the surgeon may be able to make changes to the suggested surgical plan (e.g., based on differences between the selected procedure and the surgical procedure to be performed). After confirming the surgical plan (e.g., with any changes made), the processor 104 may provide the surgical instrument suggestion 112 to indicate which surgical instruments should be loaded in a surgical tray to perform the surgical procedure (e.g., based on the availability of surgical instruments in the hospital's inventory). In some embodiments, the processor may also autoload the surgical tray with the selected surgical instruments in a surgical order (e.g., order in which the surgical instruments are to be used for performing the surgical procedure).

In some embodiments, a rendering of the patient's radiology images and mapping (e.g., acquired from the set of surgery parameters 108) with the machine learning model 110 may enable the processor 104 to display a first cut sectional view for the surgery planning. The processor may then also suggest a closest match of the previously conducted surgeries based on a comparison of the cuts and/or incisions made for the previously conducted surgeries and the first cut sectional view, allowing the surgeon to select between the various options of the previously conducted surgeries based on the similarity index(es) and a visualization of the previously conducted surgeries with respect to the surgical procedure to be performed. Additionally, in some embodiments, the availability of the previous surgeries data and planning information can be used as a training material for end users and employees.

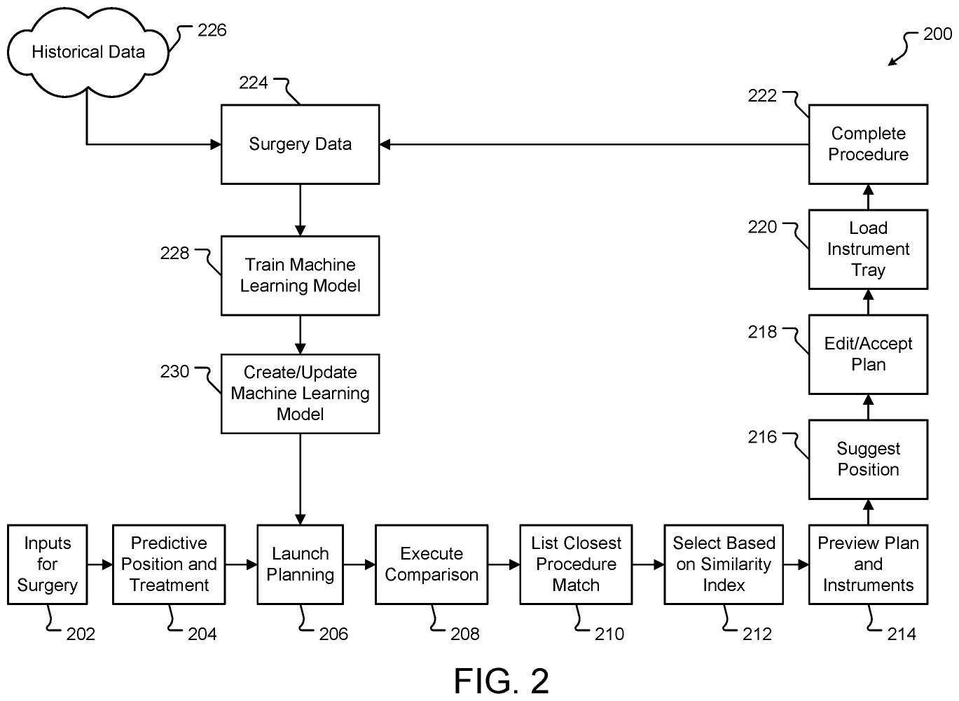

FIG. 2 is a diagram of a workflow 200 according to at least one embodiment of the present disclosure. In some examples, the workflow 200 may implement aspects of or may be implemented by aspects of FIG. 1. For example, the workflow 200 may be a more detailed view of the system 100, where a machine learning model uses inputs for a surgery to determine a surgical plan and a surgical instrument suggestion based on historical data of previously performed surgeries. In some examples, the workflow 200 may be performed by a processor described herein, such as the processor 104 as described with reference to FIG. 1.

At operation 202 of the workflow 200, one or more inputs for a given surgical procedure for a patient may be provided or received. For example, the one or more inputs may include demographic information for the patient, radiology and physiology images and data of the patient for the given surgical procedure, pathology data for the patient, or a combination thereof. At operation 204 of the workflow 200, a predictive position and treatment for the patient and given surgical procedure may be provided. At operation 206, planning of a surgical procedure for the patient may be launched. In some examples, the planning may be launched or may be based on the machine learning model as described herein. For example, the machine learning model may include or may be trained based on historical data 226 (e.g., stored in a database or cloud database) of previously performed surgeries, including surgery data 224.

Subsequently, the workflow 200 may perform operation 208 to execute a comparison between the given surgical procedure and the historical data 226 of the previously performed surgeries. At operation 210, the processor may display (e.g., via a user interface) and list the closest procedure match(es) from the previously performed surgeries that are most similar to the given surgical procedure to be performed. In some embodiments, the processor may display similarity index(es) indicating how similar each of the previously performed surgeries are to the given surgical procedure to be performed and/or how similar different aspects of the previously performed surgeries are to corresponding aspects of the given surgical procedure to be performed.

At operation 212 of the workflow 200, one of the closest procedure matches may be selected (e.g., by a surgeon or other medical provider) based on the similarity index(es). Based on the selected closest procedure, a surgical plan and instrument suggestion may be previewed and displayed by the processor to the surgeon (e.g., via a user interface) at operation 214. In some embodiments, the surgical plan may include a disease state for the patient, such as an angle, depth, and position of a targeted area in the patient to be accessed as part of the surgical procedure. Additionally, at operation 216, the processor may suggest a position of the patient for performing the given surgical procedure (e.g., to reduce load on the targeted area, fractured bone, etc.), such as on their side, on their stomach, etc.

At operation 218 of the workflow 200, the surgeon may edit and/or accept the surgical plan that is based on the selected closest procedure. For example, after making the selection, the surgeon may be able to make changes to the suggested surgical plan (e.g., based on differences between the selected procedure and the surgical procedure to be performed, differences between the patient for the given surgical procedure and the patient for which the selected procedure was performed, etc.). At operation 220, the processor may provide an output that indicates the surgical instruments to load in a surgical tray for performing the given surgical procedure for the patient based on the selected closest procedure and/or changes made to the suggested surgical plan. In some embodiments, the processor may autoload the instrument tray with the surgical tools (e.g., in a surgical order for performing the surgical procedure). Additionally or alternatively, the processor may display the surgical instruments for the surgeon to load in the surgical tray.

At operation 222, the surgeon may perform and complete the surgical procedure using the surgical instruments suggested, displayed, and/or autoloaded based on the selected closest procedure and suggested surgical plan. After completing the surgical procedure, the surgeon may provide feedback for the machine learning model (e.g., which surgical tools were or were not used, performance data for the suggested surgical plan, additional data, etc.) to further train and/or update the machine learning model. In some examples, the feedback may include surgery data 224 for the completed surgical procedure, such as 3D models, angle and position of the surgery to reach a targeted area of the patient for the surgical procedure, dimensions, annotations, treatment plans, radiology diagnostic imaging, an implant used with respect to the 3D models, etc. The surgery data 224 may also include similar information from the historical data 226 for the previously performed surgeries.

The historical data 226 and the surgery data 224 may be used to train the machine learning model at operation 228 in a continuous feedback loop to mature and continually refine the machine learning model (e.g., including performing validation and testing of the machine learning model). Accordingly, the machine learning model may be created and updated at operation 230 of the workflow 200 after training based on the historical data 226 and the surgery data 224.

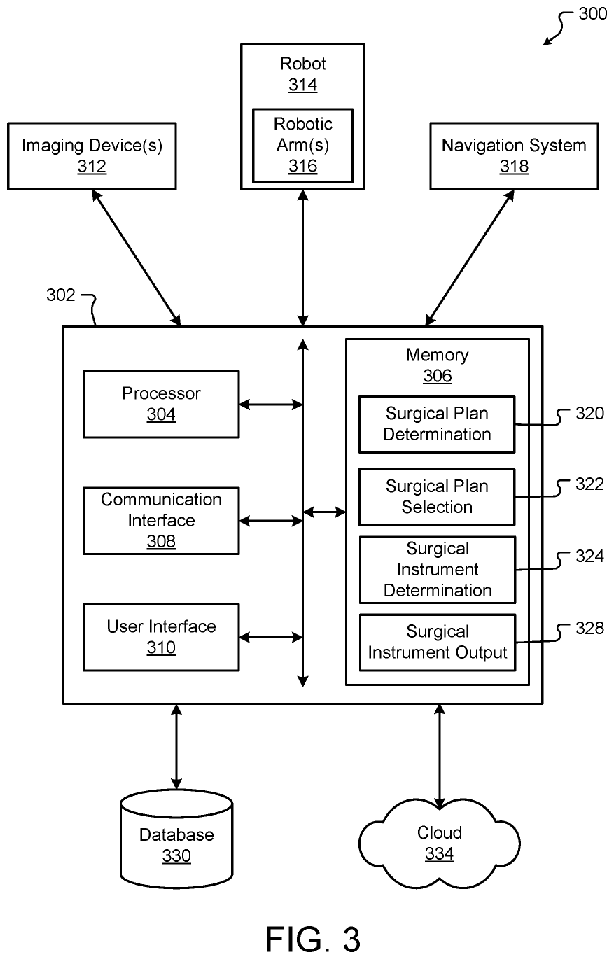

Turning to FIG. 3, a diagram of a system 300 according to at least one embodiment of the present disclosure is shown. The system 300 may be used to suggest a surgical plan and/or instrument selection for performing a surgical procedure. The system 300 comprises a computing device 302, one or more imaging devices 312, a robot 314, a navigation system 318, a database 330, and/or a cloud or other network 334. Systems according to other embodiments of the present disclosure may comprise more or fewer components than the system 300. For example, the system 300 may not include the imaging device 312, the robot 314, the navigation system 318, one or more components of the computing device 302, the database 330, and/or the cloud 334.

The computing device 302 comprises a processor 304, a memory 306, a communication interface 308, and a user interface 310. Computing devices according to other embodiments of the present disclosure may comprise more or fewer components than the computing device 302.

The processor 304 of the computing device 302 may be any processor described herein or any similar processor. The processor 304 may be configured to execute instructions stored in the memory 306, which instructions may cause the processor 304 to carry out one or more computing steps utilizing or based on data received from the imaging device 312, the robot 314, the navigation system 318, the database 330, and/or the cloud 334.

The memory 306 may be or comprise RAM, DRAM, SDRAM, other solid-state memory, any memory described herein, or any other tangible, non-transitory memory for storing computer-readable data and/or instructions. The memory 306 may store information or data useful for completing, for example, any step of the methods 400, 500, and/or 600 described herein, or of any other methods. The memory 306 may store, for example, instructions and/or machine learning models that support one or more functions of the robot 314. For instance, the memory 306 may store content (e.g., instructions and/or machine learning models) that, when executed by the processor 304, enable surgical plan determination 320, surgical plan selection 322, surgical instrument determination 324, and/or surgical instrument output 328.

The surgical plan determination 320 enables the processor 304 to receive a set of inputs for a surgical procedure for a patient and to determine one or more potential plans for the surgical procedure based at least in part on the set of inputs and a machine learning model. For example, the set of inputs for the surgical procedure may comprise patient demographic data, one or more radiological images, pathology data, or a combination thereof. Additionally, the one or more potential plans may be determined based at least in part on a disease state corresponding to the surgical procedure for a patient.

In some embodiments, the surgical plan determination 320 enables the processor 304 to compare the set of inputs for the surgical procedure with historical data of previously performed surgical procedures to determine the one or more potential plans based at least in part on a similarity index. For example, the historical data of previously performed surgical procedures may comprise procedure and instrument flow and abnormalities, radiology images and annotations, demographic information, three-dimensional anatomical models, angles, positions, dimensions, implants used with respect to the three-dimensional models, instrument availability information, treatment plans, or a combination thereof, for the previously performed surgical procedures. Additionally, the similarity index may comprise a positional coordinate correlation, a deformation coefficient, demographic information, three-dimensional model, inventory stock matching of available surgical instruments, or a combination thereof.

The surgical plan selection 322 enables the processor 304 to receive a selection of a plan from the one or more potential plans. Additionally, the surgical plan selection 322 enables the processor 304 to display (e.g., via the user interface 310) one or more similarity index values for each of the one or more potential plans, where the selection of the plan is based at least in part on the similarity index values. In some embodiments, the surgical plan selection 322 enables the processor 304 to provide a position suggestion for the surgical procedure for the patient corresponding to the plan from the selection.

In some embodiments, the surgical plan selection 322 enables the processor 304 to provide a cut sectional view for the surgical procedure for the patient based at least in part on the set of inputs and to provide one or more cut sectional views for the one or more potential plans based at least in part on historical surgical data corresponding to the one or more potential plans, where the selection of the plan from the one or more potential plans is received based at least in part on a comparison of the cut sectional view for the surgical procedure and the one or more cut sectional views for the one or more potential plans.

The surgical instrument determination 324 enables the processor 304 to determine a plurality of surgical instruments corresponding to the plan from the selection. In some embodiments, the surgical instrument determination 324 enables the processor 304 to receive one or more changes to the plan from the selection, and the plurality of surgical instruments may be determined based at least in part on the one or more changes.

The surgical instrument output 328 enables the processor 304 to provide an output that indicates the plurality of surgical instruments to load in a surgical tray. For example, the output that indicates the plurality of surgical instruments to load in the surgical tray may comprise an order (e.g., surgical order) for loading the plurality of surgical instruments in the surgical tray. In some embodiments, the surgical instrument output 328 enables the processor 304 to display (via the user interface 310) a suggestion of the plurality of surgical instruments to load in the surgical tray. Additionally or alternatively, the surgical instrument output 328 enables the processor 304 to load the plurality of surgical instruments into the surgical tray. In some embodiments, the surgical instrument output 328 enables the processor 304 to receive feedback after the surgical procedure for the patient is performed based at least in part on the output that indicates the plurality of surgical instruments to load in the surgical tray. Accordingly, the feedback may be used, in part, to train the machine learning model.

Content stored in the memory 306, if provided as in instruction, may, in some embodiments, be organized into one or more applications, modules, packages, layers, or engines. Alternatively or additionally, the memory 306 may store other types of content or data (e.g., machine learning models, artificial neural networks, deep neural networks, etc.) that can be processed by the processor 304 to carry out the various method and features described herein. Thus, although various contents of memory 306 may be described as instructions, it should be appreciated that functionality described herein can be achieved through use of instructions, algorithms, and/or machine learning models. The data, algorithms, and/or instructions may cause the processor 304 to manipulate data stored in the memory 306 and/or received from or via the imaging device 312, the robot 314, the database 330, and/or the cloud 334.

The computing device 302 may also comprise a communication interface 308. The communication interface 308 may be used for receiving image data or other information from an external source (such as the imaging device 312, the robot 314, the navigation system 318, the database 330, the cloud 334, and/or any other system or component not part of the system 300), and/or for transmitting instructions, images, or other information to an external system or device (e.g., another computing device 302, the imaging device 312, the robot 314, the navigation system 318, the database 330, the cloud 334, and/or any other system or component not part of the system 300). The communication interface 308 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 308 may be useful for enabling the device 302 to communicate with one or more other processors 304 or computing devices 302, whether to reduce the time needed to accomplish a computing-intensive task or for any other reason.

The computing device 302 may also comprise one or more user interfaces 310. The user interface 310 may be or comprise a keyboard, mouse, trackball, monitor, television, screen, touchscreen, and/or any other device for receiving information from a user and/or for providing information to a user. The user interface 310 may be used, for example, to receive a user selection or other user input regarding any step of any method described herein. Notwithstanding the foregoing, any required input for any step of any method described herein may be generated automatically by the system 300 (e.g., by the processor 304 or another component of the system 300) or received by the system 300 from a source external to the system 300. In some embodiments, the user interface 310 may be useful to allow a surgeon or other user to modify instructions to be executed by the processor 304 according to one or more embodiments of the present disclosure, and/or to modify or adjust a setting of other information displayed on the user interface 310 or corresponding thereto.

Although the user interface 310 is shown as part of the computing device 302, in some embodiments, the computing device 302 may utilize a user interface 310 that is housed separately from one or more remaining components of the computing device 302. In some embodiments, the user interface 310 may be located proximate one or more other components of the computing device 302, while in other embodiments, the user interface 310 may be located remotely from one or more other components of the computer device 302.

The imaging device 312 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 312, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 312 may be used to obtain first image data (e.g., a first image) at a first time, and a second imaging device 312 may be used to obtain second image data (e.g., a second image) at a second time after the first time. The imaging device 312 may be capable of taking a 2D image or a 3D image to yield the image data. The imaging device 312 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 312 suitable for obtaining images of an anatomical feature of a patient. The imaging device 312 may be contained entirely within a single housing, or may comprise a transmitter/emitter and a receiver/detector that are in separate housings or are otherwise physically separated.

In some embodiments, the imaging device 312 may comprise more than one imaging device 312. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 312 may be operable to generate a stream of image data. For example, the imaging device 312 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 314 may be any surgical robot or surgical robotic system. The robot 314 may be or comprise, for example, the Mazor X™ Stealth Edition robotic guidance system. The robot 314 may be configured to position the imaging device 312 at one or more precise position(s) and orientation(s), and/or to return the imaging device 312 to the same position(s) and orientation(s) at a later point in time. The robot 314 may additionally or alternatively be configured to manipulate a surgical tool (whether based on guidance from the navigation system 318 or not) to accomplish or to assist with a surgical task. In some embodiments, the robot 314 may be configured to hold and/or manipulate an anatomical element during or in connection with a surgical procedure. The robot 314 may comprise one or more robotic arms 316. In some embodiments, the robotic arm 316 may comprise a first robotic arm and a second robotic arm, though the robot 314 may comprise more than two robotic arms. In some embodiments, one or more of the robotic arms 316 may be used to hold and/or maneuver the imaging device 312. In embodiments where the imaging device 312 comprises two or more physically separate components (e.g., a transmitter and receiver), one robotic arm 316 may hold one such component, and another robotic arm 316 may hold another such component. Each robotic arm 316 may be positionable independently of the other robotic arm. The robotic arms 316 may be controlled in a single, shared coordinate space, or in separate coordinate spaces.

The robot 314, together with the robotic arm 316, may have, for example, one, two, three, four, five, six, seven, or more degrees of freedom. Further, the robotic arm 316 may be positioned or positionable in any pose, plane, and/or focal point. The pose includes a position and an orientation. As a result, an imaging device 312, surgical tool, or other object held by the robot 314 (or, more specifically, by the robotic arm 316) may be precisely positionable in one or more needed and specific positions and orientations.

The robotic arm(s) 316 may comprise one or more sensors that enable the processor 304 (or a processor of the robot 314) to determine a precise pose in space of the robotic arm (as well as any object or element held by or secured to the robotic arm).

In some embodiments, reference markers (e.g., navigation markers) may be placed on the robot 314 (including, e.g., on the robotic arm 316), the imaging device 312, or any other object in the surgical space. The reference markers may be tracked by the navigation system 318, and the results of the tracking may be used by the robot 314 and/or by an operator of the system 300 or any component thereof. In some embodiments, the navigation system 318 can be used to track other components of the system (e.g., imaging device 312) and the system can operate without the use of the robot 314 (e.g., with the surgeon manually manipulating the imaging device 312 and/or one or more surgical tools, based on information and/or instructions generated by the navigation system 318, for example).

The navigation system 318 may provide navigation for a surgeon and/or a surgical robot during an operation. The navigation system 318 may be any now-known or future-developed navigation system, including, for example, the Medtronic StealthStation™ S8 surgical navigation system or any successor thereof. The navigation system 318 may include one or more cameras or other sensor(s) for tracking one or more reference markers, navigated trackers, or other objects within the operating room or other room in which some or all of the system 300 is located. The one or more cameras may be optical cameras, infrared cameras, or other cameras. In some embodiments, the navigation system 318 may comprise one or more electromagnetic sensors. In various embodiments, the navigation system 318 may be used to track a position and orientation (e.g., a pose) of the imaging device 312, the robot 314 and/or robotic arm 316, and/or one or more surgical tools (or, more particularly, to track a pose of a navigated tracker attached, directly or indirectly, in fixed relation to the one or more of the foregoing). The navigation system 318 may include a display for displaying one or more images from an external source (e.g., the computing device 302, imaging device 312, or other source) or for displaying an image and/or video stream from the one or more cameras or other sensors of the navigation system 318. In some embodiments, the system 300 can operate without the use of the navigation system 318. The navigation system 318 may be configured to provide guidance to a surgeon or other user of the system 300 or a component thereof, to the robot 314, or to any other element of the system 300 regarding, for example, a pose of one or more anatomical elements, whether or not a tool is in the proper trajectory, and/or how to move a tool into the proper trajectory to carry out a surgical task according to a preoperative or other surgical plan.

The database 330 may store information that correlates one coordinate system to another (e.g., one or more robotic coordinate systems to a patient coordinate system and/or to a navigation coordinate system). The database 330 may additionally or alternatively store, for example, one or more surgical plans (including, for example, pose information about a target and/or image information about a patient's anatomy at and/or proximate the surgical site, for use by the robot 314, the navigation system 318, and/or a user of the computing device 302 or of the system 300); one or more images useful in connection with a surgery to be completed by or with the assistance of one or more other components of the system 300; and/or any other useful information. The database 330 may be configured to provide any such information to the computing device 302 or to any other device of the system 300 or external to the system 300, whether directly or via the cloud 334. In some embodiments, the database 330 may be or comprise part of a hospital image storage system, such as a picture archiving and communication system (PACS), a health information system (HIS), and/or another system for collecting, storing, managing, and/or transmitting electronic medical records including image data.

The cloud 334 may be or represent the Internet or any other wide area network. The computing device 302 may be connected to the cloud 334 via the communication interface 308, using a wired connection, a wireless connection, or both. In some embodiments, the computing device 302 may communicate with the database 330 and/or an external device (e.g., a computing device) via the cloud 334.

The system 300 or similar systems may be used, for example, to carry out one or more aspects of any of the methods 400, 500, and/or 600 described herein. The system 300 or similar systems may also be used for other purposes.

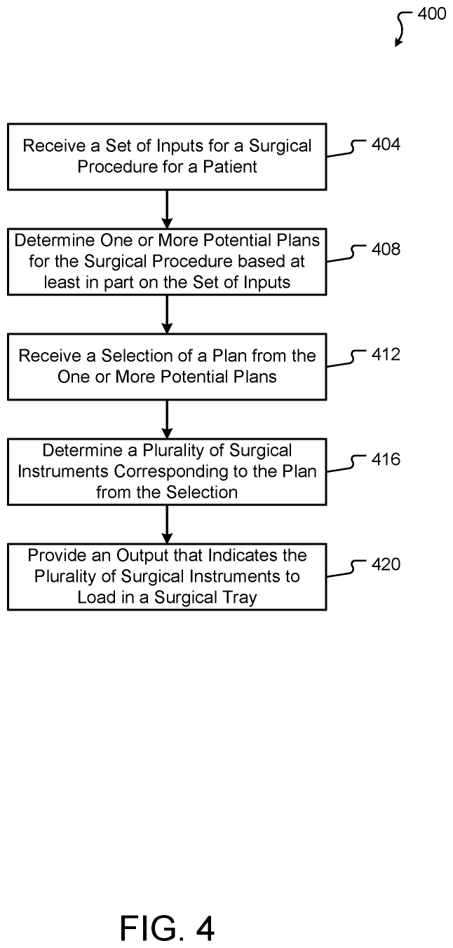

FIG. 4 depicts a method 400 that may be used, for example, to suggest a surgery plan and a surgical instrument selection.

The method 400 (and/or one or more steps thereof) may be carried out or otherwise performed, for example, by at least one processor. The at least one processor may be the same as or similar to the processor(s) 304 of the computing device 302 described above. The at least one processor may be part of a robot (such as a robot 314) or part of a navigation system (such as a navigation system 318). A processor other than any processor described herein may also be used to execute the method 400. The at least one processor may perform the method 400 by executing elements stored in a memory such as the memory 306. The elements stored in the memory and executed by the processor may cause the processor to execute one or more steps of a function as shown in method 400. One or more portions of a method 400 may be performed by the processor executing any of the contents of memory, such as a surgical plan determination 320, a surgical plan selection 322, a surgical instrument determination 324, and/or a surgical instrument output 328.

The method 400 comprises receiving a set of inputs for a surgical procedure for a patient (step 404). For example, the set of inputs for the surgical procedure may comprise patient demographic data, one or more radiological images, pathology data, or a combination thereof.

The method 400 also comprises determining one or more potential plans for the surgical procedure based at least in part on the set of inputs (step 408). In some embodiments, the one or more potential plans are determined based at least in part on a disease state corresponding to the surgical procedure for a patient.

The method 400 also comprises receiving a selection of a plan from the one or more potential plans (step 412). In some embodiments, a position suggestion may be provided for the surgical procedure for the patient corresponding to the plan from the selection. Additionally, a cut sectional view for the surgical procedure for the patient may be provided based at least in part on the set of inputs, and one or more cut sectional views for the one or more potential plans may also be provided based at least in part on historical surgical data corresponding to the one or more potential plans. Accordingly, in some embodiments, the selection of the plan from the one or more potential plans may be received based at least in part on a comparison of the cut sectional view for the surgical procedure and the one or more cut sectional views for the one or more potential plans.

The method 400 also comprises determining a plurality of surgical instruments corresponding to the plan from the selection (step 416). In some embodiments, one or more changes to the plan from the selection may be received, and the plurality of surgical instruments may be determined based at least in part on the one or more changes.

The method 400 also comprises providing an output that indicates the plurality of surgical instruments to load in a surgical tray (step 420). In some examples, the output that indicates the plurality of surgical instruments to load in the surgical tray may comprise an order (e.g., surgical order) for loading the plurality of surgical instruments in the surgical tray. In some embodiments, providing the output may comprise displaying, via a user interface, a suggestion of the plurality of surgical instruments to load in the surgical tray. Additionally or alternatively, providing the output may comprise loading the plurality of surgical instruments into the surgical tray.

The present disclosure encompasses embodiments of the method 400 that comprise more or fewer steps than those described above, and/or one or more steps that are different than the steps described above.

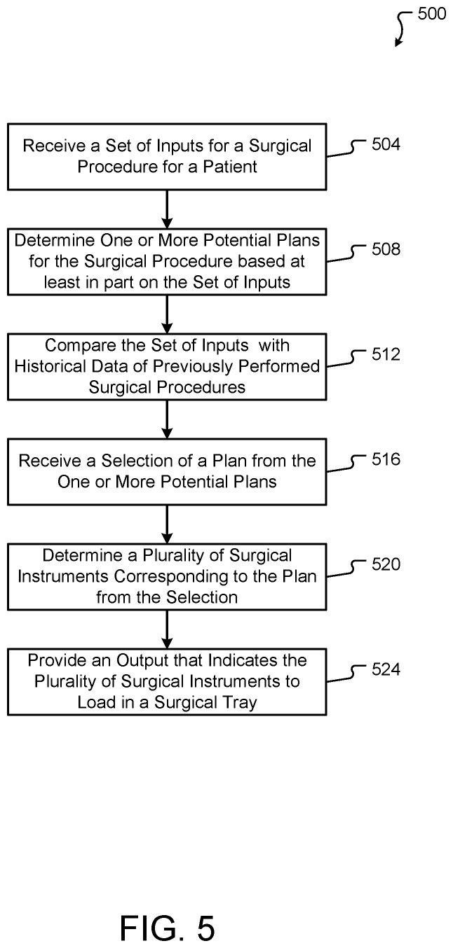

FIG. 5 depicts a method 500 that may be used, for example, to compare a given surgical procedure with previously performed surgical procedures.

The method 500 (and/or one or more steps thereof) may be carried out or otherwise performed, for example, by at least one processor. The at least one processor may be the same as or similar to the processor(s) 304 of the computing device 302 described above. The at least one processor may be part of a robot (such as a robot 314) or part of a navigation system (such as a navigation system 318). A processor other than any processor described herein may also be used to execute the method 500. The at least one processor may perform the method 500 by executing elements stored in a memory such as the memory 306. The elements stored in memory and executed by the processor may cause the processor to execute one or more steps of a function as shown in method 500. One or more portions of a method 500 may be performed by the processor executing any of the contents of memory, such as a surgical plan determination 320, a surgical plan selection 322, a surgical instrument determination 324, and/or a surgical instrument output 328.

The method 500 comprises receiving a set of inputs for a surgical procedure for a patient (step 504). The method 500 also comprises determining one or more potential plans for the surgical procedure based at least in part on the set of inputs (step 508). Steps 504 and 508 may implement similar aspect of steps 404 and 408, respectively, as described with reference to FIG. 4.

In some embodiments, as described herein, the one or more potential plans may further be determined based at least in part on a machine learning model. For example, the method 500 also comprises comparing (e.g., based on the machine learning model) the set of inputs for the surgical procedure with historical data of previously performed surgical procedures to determine the one or more potential plans based at least in part on a similarity index (step 512). In some embodiments, the historical data of previously performed surgical procedures may comprise procedure and instrument flow and abnormalities, radiology images and annotations, demographic information, three-dimensional anatomical models, angles, positions, dimensions, implants used with respect to the three dimensional models, instrument availability information, treatment plans, or a combination thereof, for the previously performed surgical procedures. Additionally, the similarity index may comprise a positional coordinate correlation, a deformation coefficient, demographic information, three-dimensional model, inventory stock matching of available surgical instruments, or a combination thereof.

The method 500 also comprises receiving a selection of a plan from the one or more potential plans (step 516). Step 516 may implement aspects of step 412 as described with reference to FIG. 4. Additionally, in some embodiments, one or more similarity index values for each of the one or more potential plans may be displayed (e.g., via a user interface), and the selection of the plan may be received based at least in part on the one or more similarity index values.

The method 500 also comprises determining a plurality of surgical instruments corresponding to the plan from the selection (step 520). The method 500 also comprises providing an output that indicates the plurality of surgical instruments to load in a surgical tray (step 524). Steps 520 and 524 may represent aspects of steps 416 and 420 as described with reference to FIG. 4.

The present disclosure encompasses embodiments of the method 500 that comprise more or fewer steps than those described above, and/or one or more steps that are different than the steps described above.

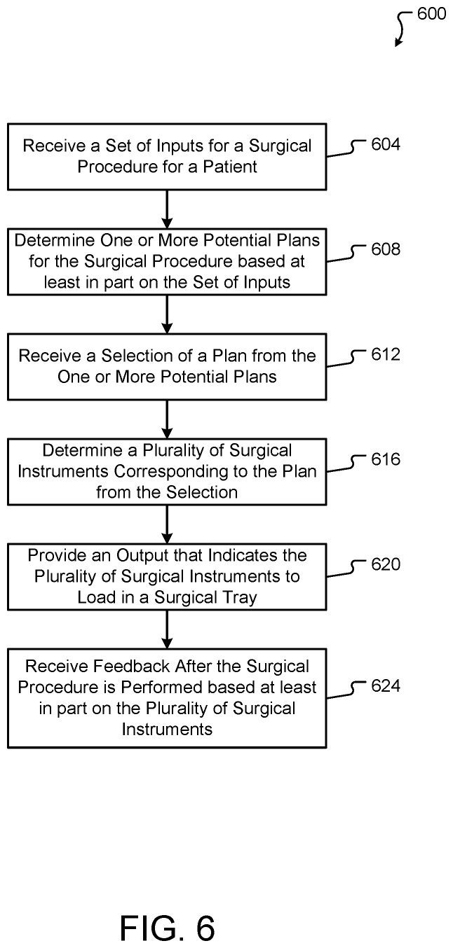

FIG. 6 depicts a method 600 that may be used, for example, to update a machine learning model for suggesting a surgery plan and a surgical instrument selection based on a feedback loop.

The method 600 (and/or one or more steps thereof) may be carried out or otherwise performed, for example, by at least one processor. The at least one processor may be the same as or similar to the processor(s) 304 of the computing device 302 described above. The at least one processor may be part of a robot (such as a robot 314) or part of a navigation system (such as a navigation system 318). A processor other than any processor described herein may also be used to execute the method 600. The at least one processor may perform the method 600 by executing elements stored in a memory such as the memory 306. The elements stored in memory and executed by the processor may cause the processor to execute one or more steps of a function as shown in method 600. One or more portions of a method 600 may be performed by the processor executing any of the contents of memory, such as a surgical plan determination 320, a surgical plan selection 322, a surgical instrument determination 324, and/or a surgical instrument output 328.

The method 600 comprises receiving a set of inputs for a surgical procedure for a patient (step 604). The method 600 also comprises determining one or more potential plans for the surgical procedure based at least in part on the set of inputs (e.g., and a machine learning model as described herein) (step 608). The method 600 also comprises receiving a selection of a plan from the one or more potential plans (step 612). The method 600 also comprises determining a plurality of surgical instruments corresponding to the plan from the selection (step 616). The method 600 also comprises providing an output that indicates the plurality of surgical instruments to load in a surgical tray (step 620). Steps 604, 608, 612, 616, and 620 may represent steps 404, 408, 412, 416, and 420, respectively, as described with reference to FIG. 4 and steps 504, 508, 516, 520, and 524, respectively, as described with reference to FIG. 5.

The method 600 also comprises receiving feedback after the surgical procedure for the patient is performed based at least in part on the output that indicates the plurality of surgical instruments to load in the surgical tray (step 624). In some embodiments, the feedback may be used, in part, to train the machine learning model.

The present disclosure encompasses embodiments of the method 600 that comprise more or fewer steps than those described above, and/or one or more steps that are different than the steps described above.

As noted above, the present disclosure encompasses methods with fewer than all of the steps identified in FIGS. 4, 5, and 6 (and the corresponding description of the methods 400, 500, and 600), as well as methods that include additional steps beyond those identified in FIGS. 4, 5, and 6 (and the corresponding description of the methods 400, 500, and 600). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for suggesting a surgery plan and a surgical instrument selection, comprising:
a processor; and
a memory storing data for processing by the processor, the data, when processed, causes the processor to:
receive a set of inputs for a surgical procedure for a patient;
determine one or more potential plans for the surgical procedure based at least in part on the set of inputs and a machine learning model;
receive a selection of a plan from the one or more potential plans;
determine a plurality of surgical instruments corresponding to the plan from the selection; and
provide an output that indicates the plurality of surgical instruments to load in a surgical tray.

2. The system of claim 1, wherein the data stored in the memory that, when processed causes the processor to provide the output that indicates the plurality of surgical instruments to load in the surgical tray further causes the system to:
display, via a user interface, a suggestion of the plurality of surgical instruments to load in the surgical tray.

3. The system of claim 1, wherein the data stored in the memory that, when processed causes the processor to provide the output that indicates the plurality of surgical instruments to load in the surgical tray further causes the system to:
load the plurality of surgical instruments into the surgical tray.

4. The system of claim 1, wherein the data stored in the memory that, when processed causes the processor to determine the one or more potential plans for the surgical procedure further causes the system to:
compare the set of inputs for the surgical procedure with historical data of previously performed surgical procedures to determine the one or more potential plans based at least in part on a similarity index.

5. The system of claim 4, wherein the memory stores further data for processing by the processor that, when processed, causes the processor to:
display, via a user interface, one or more similarity index values for each of the one or more potential plans.

6. The system of claim 4, wherein the historical data of previously performed surgical procedures comprises procedure and instrument flow and abnormalities, radiology images and annotations, demographic information, three-dimensional anatomical models, angles, positions, dimensions, implants used with respect to the three-dimensional models, instrument availability information, treatment plans, or a combination thereof, for the previously performed surgical procedures.

7. The system of claim 4, wherein the similarity index comprises a positional coordinate correlation, a deformation coefficient, demographic information, three-dimensional model, inventory stock matching of available surgical instruments, or a combination thereof.

8. The system of claim 1, wherein the memory stores further data for processing by the processor that, when processed, causes the processor to:
provide a position suggestion for the surgical procedure for the patient corresponding to the plan from the selection.

9. The system of claim 1, wherein the memory stores further data for processing by the processor that, when processed, causes the processor to:
receive one or more changes to the plan from the selection, wherein the plurality of surgical instruments to load in the surgical tray are determined based at least in part on the one or more changes.

10. The system of claim 1, wherein the memory stores further data for processing by the processor that, when processed, causes the processor to:
provide a cut sectional view for the surgical procedure for the patient based at least in part on the set of inputs; and
provide one or more cut sectional views for the one or more potential plans based at least in part on historical surgical data corresponding to the one or more potential plans, wherein the selection of the plan from the one or more potential plans is received based at least in part on a comparison of the cut sectional view for the surgical procedure and the one or more cut sectional views for the one or more potential plans.

11. The system of claim 1, wherein the memory stores further data for processing by the processor that, when processed, causes the processor to:
receive feedback after the surgical procedure for the patient is performed based at least in part on the output that indicates the plurality of surgical instruments to load in the surgical tray.

12. The system of claim 11, wherein the feedback is used, in part, to train the machine learning model.

13. The system of claim 1, wherein the one or more potential plans are determined based at least in part on a disease state corresponding to the surgical procedure for a patient.

14. The system of claim 1, wherein the set of inputs for the surgical procedure comprises patient demographic data, one or more radiological images, pathology data, or a combination thereof.

15. The system of claim 1, wherein the output that indicates the plurality of surgical instruments to load in the surgical tray comprises an order for loading the plurality of surgical instruments in the surgical tray.

16. A system for suggesting a surgery plan and a surgical instrument selection, comprising:
a processor; and
a memory storing data for processing by the processor, the data, when processed, causes the processor to:
receive a set of inputs for a surgical procedure for a patient;
determine one or more potential plans for the surgical procedure based at least in part on the set of inputs;
receive a selection of a plan from the one or more potential plans;
determine a plurality of surgical instruments corresponding to the plan from the selection; and
provide an output that indicates the plurality of surgical instruments to load in a surgical tray.

17. The system of claim 16, wherein the data stored in the memory that, when processed causes the processor to determine the one or more potential plans for the surgical procedure further causes the system to:
compare the set of inputs for the surgical procedure with historical data of previously performed surgical procedures to determine the one or more potential plans based at least in part on a similarity index.

18. The system of claim 17, wherein the memory stores further data for processing by the processor that, when processed, causes the processor to:
display, via a user interface, one or more similarity index values for each of the one or more potential plans.

19. A system for suggesting a surgery plan and a surgical instrument selection, comprising:
a processor; and
a memory storing data for processing by the processor, the data, when processed, causes the processor to:
receive a set of inputs for a surgical procedure for a patient;
determine one or more potential plans for the surgical procedure based at least in part on the set of inputs;
receive a selection of a plan from the one or more potential plans;
determine a plurality of surgical instruments corresponding to the plan from the selection; and
load the plurality of surgical instruments into a surgical tray.

20. The system of claim 19, wherein the plurality of surgical instruments is loaded in the surgical tray according to an order for performing the surgical procedure for the patient based at least in part on the plan from the selection.
